(19) United States
(12) Patent Application Publication
(10) Pub. No.: US 2004/0122065 A1
(43) Pub. Date: Jun. 24, 2004

(54) Pharmaceutical compositions and dosage forms for buccal and sublingual delivery of tizanidine and methods of administering tizanidine sublingually or buccally

(76) Inventors: E. Itzhak Lerner, Petach Tikva (IL); Moshe Flashner-Barak, Petach Tikva (IL); Vered Rosenberger, Jerusalem (IL)

Correspondence Address: KENYON & KENYON, ONE BROADWAY, NEW YORK, NY, 10004, US

(21) Appl. No.: 10/699,991

(22) Filed: Nov. 3, 2003

Related U.S. Application Data
(60) Provisional application No. 60/425,326, filed on Nov. 12, 2002.

(51) Int. Cl. A61K 31/433

(52) U.S. Cl. 514/361

(57) ABSTRACT

Sublingual and buccal administration of the muscle spasm suppressor tizanidine increase its bioavailability by avoiding first-pass metabolism in the liver and reduce the inter-patient variation in bioavailability.

### CROSS-REFERENCE TO RELATED APPLICATION

This application claims the benefit of U.S. provisional application No. 60/425,326, filed on Nov. 12, 2002, the disclosure of which is entirely incorporated by reference herein.

### FIELD OF THE INVENTION

The present invention relates to anti-spasmodic agents and, more particularly, to improved methods of administration and dosage forms of tizanidine.

### BACKGROUND OF THE INVENTION

5-Chloro-N-(4,5-dihydro-1H-imidazol-2-yl)-2,1,3-benzothiadiazol-4-amine, whose non-systemic chemical name is tizanidine and whose chemical formula is

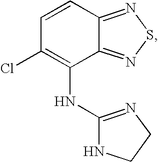

is a centrally acting α₂-adrenergic receptor agonist. It is indicated for suppression of muscle spasms occurring with a wide range of etiologies: spasticity in general (refs. 2-5); muscle spasms caused by multiple sclerosis (refs. 6-8); spinal chord injury (refs. 9, 10); and brain injury (refs. 11, 12). Tizanidine also has been evaluated for treatment of chronic headache with positive results (refs. 13-16).

Tizanidine hydrochloride is commercially available in an immediate release oral tablet formulation under the brand name Zanaflex™. Zanaflex™ is a conventional oral dosage form whose active ingredient is absorbed into the bloodstream through the mucosa lining the stomach and small intestine. This route of administration is referred to in this disclosure as enteric delivery. Pharmaceutical compositions and dosage forms that are taken through the mouth alternatively may be held there while the active ingredient is released. Depending upon the active ingredient, it may be absorbed through the mucosa lining the mouth.

The bioavailability of tizanidine is highly variable from patient to patient, necessitating titration of the dose level on an individual basis. Tizanidine can cause hepatic toxicity, which is another reason that the dosage and plasma level of tizanidine should be carefully controlled (refs. 1, 18). Tizanidine that is administered in Zanaflex™ is essentially completely absorbed by the intestinal mucosa but the bioavailability of tizanidine is only about 40% due to first-pass hepatic metabolism to metabolites, all of which appear to be pharmacologically inactive. Alternative routes of administration that do not involve gastric absorption will by-pass first-pass metabolism in the liver. There are many such alternative routes, namely, administration in ophthalmic preparations, intravenous, intramuscular or subcutaneous injection, inhalation, transdermal and topical administration, and buccal and sublingual administration. Sublingual administration involves the patient holding a pharmaceutical composition or dosage form under their tongue while the drug diffuses into the mouth, through the mucosa lining the mouth and from there into the bloodstream. In buccal administration, the patient holds the pharmaceutical composition or dosage form between their cheek and gum instead of under the tongue. Like the other alternatives to gastric delivery, buccal and sublingual administration might conceivably raise the bioavailability of tizanidine by avoiding first pass hepatic metabolism. However, only a few drugs can be given successfully by this route. Remington's Pharmaceutical Sciences 670 (Mack Publishing: Easton, Pa. 1980). The drug must be rapidly absorbed by the oral mucosa or saliva will wash it out of the oral cavity. Moreover, tizanidine which is only slightly soluble in water and methanol becomes less soluble with increasing pH (ref 1). Saliva is approximately pH neutral or slightly basic. Its pH is much higher than that of gastric fluid in which known tizanidine tablets, which are swallowed, dissolve. A reduction in bioavailability due to the low solubility of tizanidine in the pH range of the mouth could reduce or overwhelm any increase in bioavailability that might otherwise be realized by absorbing the drug through the mouth.

In view of the foregoing, it will be appreciated that improvement in the methods of administering tizanidine so as to increase the bioavailability of the drug and reduce variability in dosing would be highly desirable.

### LIST OF REFERENCES CITED

1 Physicians Desk Reference 55th ed. 670-673 (Medical Economics Co.: Montvale, N.J. 2001).

2. Abbruzzese, G., “The Medical Management of Spasticity” Eur. J. Neurol., 2002, Suppl. 1,30-34, and 53-61.

3. Kita, M. and Goodkin, D. E., “Drugs Used to Treat Spasticity” Drugs, 2000, 59(3), 487-95.

4. Groves, L., et. al., “Tizanidine Treatment of Spasticity: A Meta Analysis of Controlled, Double-blind, Comparative Studies with Baclofen and Diazepam” Adv. Ther., 1998, 15(4), 241-51.

5. Milanov, I., and Georgiev, D., “Mechanisms of Tizanidine Action on Spasticity” Acta. Neurol. Scand., 1994, 89(4), 274-79).

6. Schapiro, R. T., “Management of Spasticity, Pain, And Paroxysmal Phenomena in Multiple Sclerosis” Curr. Neurosci. Rep., 2001, 1(3), 299-302.

7. Nance, P. W., et. al. “Relationship of The Antispasticity Effect of Tizanidine to Plasma Concentration in Patients with Multiple Sclerosis” Arch. Neurol., 1997, 54(6), 731-36.

8. Smith, C., et. al., “Tizanidine Treatment of Spasticity Caused by Multiple Sclerosis: Results of a Double-blind, Placebo Controlled Trial. US Tizanidine Study Group” Neurology, 1994, 44(11 Suppl. 9), S34-43).

9. Nance, P. W. et. al., “Efficacy And Safety of Tizanidine in The Treatment of Spasticity in Patients with Spinal Cord Injury. North American Tizanidine Study Group” Neurology, 1994, 44(11 suppl. 9), S44-52.

10. Burchiel, K. J. and Hsu, F. P., “Pain And Spasticity after Spinal Cord Injury: Mechanisms And Treatment” Spine, 2001, 26(24 suppl.), S146-60).

11. Gelber, D. A. et. al., “Open-label Dose-titration Safety and Efficacy Study of Tizanidine Hydrochloride in The Treatment of Spasticity Associated with Chronic Stroke” Stroke, 2001, 32(8) 1841-6.

12. Meythaler, J, M. et. al., “Prospective Assessment of Tizanidine for Spasticity Due to Acquired Brain Injury” Arch. Phys. Med. Rehabil., 2001, 82(9), 1159-63).

13. Smith, T. R., “Low-dose Tizanidine with Nonsteroidal Anti-inflammatory Drugs for Detoxification from Analgesic Rebound Headache, Headache, 2002, 42(3), 175-7.

14. Saper, J. R., et. al., “An Open-label Dose-titration Study of The Efficacy And Tolerability of Tizanidine Hydrochloride Tablets in The Prophylaxis of Chronic Daily Headache, Headache, 2001, 41(4), 357-68.

15. Murros, K., et. al., “Modified Release Formulation of Tizanidine in Chronic Tension-type Headache”, Headache, 2000, 40(8), 633-7.

16. D'Alessandro, R., “Tizanidine for Chronic Cluster Headache” Arch. Neurol., 1996, 53(11) 1093.

17. Roberts, R. C. “Pharmacokinetics and Pharmacodynamics of Tizanidine” Neurology 1994, 44 (11 suppl 9), S29-31.

18. de-Graff, E. M., et. al., “A Case of Tizanidine-induced Hepatic Injury, J. Hepatol., 1996, 25(5), 772-3.

19. Patel, M. V. and Chen, F. J., “Solid Carriers for Improved Delivery of Active Ingredients in Pharmaceutical Compositions” International Publication No. WO 01/037808.

20. Chen, F. J. and Patel, M. V., “Emulsion Compositions for Polyfunctional Active Ingredients, International Publication No. WO 01/028555.

### BRIEF DESCRIPTION OF THE FIGURES

FIG. 1: is a perspective view of a multicompression tablet having a core surrounded by an annular body in accordance with a preferred dosage form embodiment of the invention.

### SUMMARY OF THE INVENTION

The foregoing objects of the invention are accomplished and the shortcomings associated with prior art techniques for administration of the muscle spasm suppressor tizanidine are overcome with the present invention by effectively administering tizanidine buccally or sublingually.

In accordance with one method aspect of the invention, the bioavailability of tizanidine is increased by sublingual or buccal administration relative to administration of a comparable dosage of tizanidine in a conventional enteral dosage form. The increase in bioavailability as measured by the area under the curve extrapolated to infinity of the blood stream concentration of tizanidine can be increased by as much as 10% or more.

The bioavailability of tizanidine when administered by conventional enteral dosage forms is highly variable from patient to patient. In accordance with another method aspect of the invention sublingual or buccal administration of tizanidine reduces the inter-patient variability of the bioavailability of tizanidine. Sublingual or buccal administration of tizanidine in accordance with this invention to a population of patients can reduce the relative standard deviation of the bloodstream concentration of tizanidine of the patient population by 10% or more.

Dosage forms especially adapted for sublingual and buccal administration of tizanidine are provided by this invention as well. Tizanidine has lower solubility in saliva that in gastric fluid due to the difference in pH. One of the dosage form embodiments includes an acidulant that acidifies the pH in the local environment in the sublingual or buccal cavity to accelerate release of tizanidine into the bloodstream. Yet further dosage form embodiments of this invention enable timed release of the tizanidine that is slow enough to avoid accumulation of tizanidine in the mouth, yet rapid enough to be acceptable to a patient who holds the dosage form in the mouth while the tizanidine is being released. Among these dosage forms is a liquid that congeals in the mouth and conforms to the space under the tongue or between the cheek and gum to provide a large contact surface area and comfortable feel.

### DETAILED DESCRIPTION OF THE INVENTION

We have discovered that buccal and sublingual administration of tizanidine (also “the drug”) improve the drug's bioavailability and greatly diminish absorption variability between patients.

Accordingly, one aspect of the present invention is a method of treating muscle spasms by buccal or sublingual administration of tizanidine. Tizanidine can be administered in any pharmaceutical composition or dosage form that can be held in the mouth for an extended period of time and permits diffusion or erosion of the drug into the mouth cavity where it can be absorbed through the mucosa lining of the mouth. Such dosage forms include tablets, lozenges, troches, pastilles, pills, viscous liquids, pastes, sprays, drops, gels, patches and the like.

There are unique challenges to administering drugs buccally and sublingually, which the skilled formulation scientist can address and overcome using known techniques. To further enable the formulation chemist in overcoming these challenges, the invention provides pharmaceutical compositions and dosage forms especially adapted for buccal and sublingual administration of tizanidine, described below.

One of the challenges of sublingual and buccal drug administration involves controlling the rate of release of the drug. If the drug is released more rapidly than it can be absorbed through the mouth, its concentration will increase in the saliva and it will be swallowed, whereupon it will be absorbed in the gut as though it were given in a conventional oral dosage form. Thus, it will be appreciated that control of the release rate will affect the drug's bioavailability and variability in absorption between patients.

Preferably, in the treatment method of the invention, tizanidine is 80% released or more within 20 minutes after administration of the drug, since most patients do not like to hold a tablet or lozenge under the tongue for longer periods. More preferably the composition or dosage form releases 80% or more tizanidine in 5 minutes or less.

Another aspect of this invention is a method of increasing the bioavailability of tizanidine by means of buccal or sublingual administration. Bioavailability refers to the proportion of the drug administered that reaches the physiological site where the drug exerts its therapeutic effect, which is generally regarded as the blood stream for many drugs, and is so regarded in this disclosure for tizanidine. The bioavailability of a drug is most readily expressed as the concentration of the drug (or in some instances of active metabolites) in the blood plasma integrated over time. This quantity is commonly referred to as the “area under the curve” or “AUC”. The bioavailability of a drug administered in different formulations and by different routes can be compared by comparing the AUCs from patients that have taken both formulations at different times. According to good laboratory practice for a comparative study of different formulations on humans, a population of test subjects is divided into two groups equal in number. Under controlled conditions, one group is administered the drug in one formulation while the other group is administered the other formulation. Their blood plasma concentrations of the drug are monitored for a period of time and the data is collected and analyzed. A “wash out” period is then allowed to pass during which the drug is eliminated from their bodies so that a second phase of the study will begin with a zero blood plasma concentration of the drug. In the second phase, the group that received the first formulation of the drug is administered the drug in the second formulation, the group that received the second formulation is administered the first formulation, and monitoring, data collection and analysis are repeated. Administering both formulations to the same population minimizes error in comparison of bioavailability due to age, sex and individual physiological factors.

As a practical matter, the blood plasma concentration of test subjects is measured over a limited amount of time and with limited frequency to minimize discomfort to the test subjects. Integration of the blood plasma concentration of the drug over that limited time period affords the AUC for an individual. The blood plasma concentration of the drug in an individual will not necessarily have fallen to zero at the end of the monitoring period. Therefore, the AUC will underestimate the relative bioavailability of the drug for that individual. Data analysis software adapted for analyzing the results of clinical studies is commercially available. Such software is able to extrapolate the blood plasma concentration curve beyond the monitoring period based upon the shape of the curve during the monitoring period. The area under the extrapolated portion of the curve can be determined by integration and that area added to the area determined during the monitoring period to arrive at the area under the curve extrapolated to infinity or “AUC_{inf}” AUC_{inf} provides a more accurate measure of relative bioavailability than AUC when the monitoring is ceased before the drug has been substantially completely eliminated.

Buccal or sublingual administration of tizanidine according to this invention preferably increases the drug's bioavailability 10% or more as determined by comparison of the AUC_{inf} of the particular composition or dosage form used with the AUC_{inf} for patient(s) who swallow a conventional oral dosage form containing an equivalent dosage of tizanidine. Alternatively, the increase in bioavailability can be determined against a dosage form of different strength provided the difference is taken into account. More preferably, sublingual or buccal administration according to the invention increases the drug's bioavailability by 20% or more.

An equivalent dosage is one that contains approximately the same number of millimoles of tizanidine, regardless of whether differing weights of the active ingredient are added to the formulations to compensate for use of tizanidine in free base form, the use of a different salt anion or different states of solvation. The increase in bioavailability is referenced against an immediate release orally administered dosage form and in particular against Zanaflex™, as described in ref 1, above, since it is known that Zanaflex™ is essentially completely absorbed. Therefore, Zanaflex™ is the highest benchmark of which we are aware against which to reference improvement in bioavailability consistent with the knowledge of those in the art prior to this invention. In addition to tizanidine, commercially available Zanaflex™ contains colloidal silicon dioxide, stearic acid, microcrystalline cellulose and anhydrous lactose.

In the methods of the invention, tizanidine is preferably administered in individual doses containing from about 2 mg to about 8 mg, more preferably from about 2 mg to about 4 mg, of tizanidine based on the weight of the free base regardless whether it is administered as the free base or in a salt form. The individual doses are preferably administered at 6 to 8 hour intervals during the day up to a daily cumulative dose of from about 4 mg to about 36 mg, more preferably from about 8 to about 24 mg.

In yet another aspect, the invention provides a method of reducing variations in the blood plasma levels of tizanidine between individuals in a patient population by buccal or sublingual administration of tizanidine. A patient population includes any group of people who suffer from muscle spasms and are identifiable as a group because they share a common nexus. In addition to groups of patients whose nexus is that their muscle spasms have a common etiology, a population may refer to a group of patients who are under the care of the same doctor or healthcare provider or receive treatment for their muscle spasms at the same health care facility.

Generally, there are many ways to analyze variations in a population statistically. The most straightforward circumstance is analysis of variations in a single parameter over a population. Variation in the parameter can be quantified by calculation of the standard deviation (s.d.) or variance (s.d.²) of the parameter over the population. Relative standard deviation (r.s.d.) is the standard deviation of a parameter divided by the average value of the parameter for the population. The r.s.d. allows meaningful comparison of the degree of variation in a parameter between populations. In accordance with the present invention, the improvement in consistency of absorption achieved by administering tizanidine buccally or sublingually is reflected in a lower relative standard deviation of AUC_{inf} for a population that has been administered tizanidine sublingually or buccally than the r.s.d. of a population that has been administered tizanidine in a conventional oral dosage form that was swallowed. Preferably, the r.s.d. of the population that was administered tizanidine buccally or sublingually is about 10% lower, more preferably about 20% lower and most preferably about 30% lower. The two populations being compared preferably comprise the same individuals who have received tizanidine via these routes at different times, with a washout period separating the administrations.

All but one of the ten test subjects who completed our study that is reported in the Example showed increased bioavailability of tizanidine by sublingual administration. If 10% or more of patients who are not responding well to orally administered tizanidine and are switched to sublingual or buccal treatment show improved response, that is an indication that the variation in a population comprising those patients and patients who respond well to conventional oral treatment (who are not switched) is reduced. Therefore, in accordance with this invention, medical records or observations showing improved muscle spasm suppression in more than 10% of patients who changed from an oral to a sublingual or buccal dosage form also indicates a reduction in the variability of response between patients of a doctor or health care facility.

The methods of the invention can be practiced using any pharmaceutical composition or dosage form containing tizanidine that is appropriate for sublingual or buccal administration. Discrete dosage forms like tablets, capsules and the like preferably contain doses of from about 2 mg to about 8 mg, more preferably from about 2 mg to about 4 mg, of tizanidine based on the weight of the free base.

Appropriate compositions and dosage forms are prepared with nontoxic pharmaceutically acceptable excipients. The excipients are known to those skilled in the preparation of buccal and sublingual dosage forms. Ingredients and exemplary formulations may be found in Remington's Pharmaceutical Sciences 16th ed. (Mack Publishing 1980). The patent literature also contains many disclosures of buccal and sublingual formulations, including U.S. Pat. Nos. 4,020,558; 4,229,447; 3,972,995; 3,870,790; 3,444,858; 2,698,822; 3,632,743, all of which are incorporated herein by reference in their entirety.

Excipients that are commonly formulated into buccal and sublingual dosage forms include, maltodextrin, colloidal silicon dioxide, starch, starch syrup, sugar and α-lactose. Conventional methods of processing active ingredients and excipients into pharmaceutical compositions and dosage forms for buccal and sublingual administration are well known to the skilled formulation specialist.

Preferred pharmaceutical compositions and dosage forms release at least about 80% of the tizanidine within about twenty minutes of administration, more preferably within about 5 minutes of administration.

A further aspect of the invention provides compositions and dosage forms especially adapted for buccal and sublingual administration of tizanidine. Tizanidine is absorbed better in the acid environment of the stomach than in the neutral environment of the mouth. Acidifying the saliva, preferably to a pH between 2 and 7, improves the absorption of tizanidine.

Accordingly, preferred embodiments of the compositions and dosage forms of the present invention are able to acidify the local environment in the sublingual cavity or buccal cavity during the desired drug release period. Such dosage forms contain an effective acidifying amount of an acidulant. An acidulant is an excipient that acidifies the local environment around the dosage form or composition after it has been put in the patient's mouth. It need not acidify the saliva in all regions of the sublingual or buccal cavity to be effective, only the saliva that provides direct fluid communication between the surface of the dosage form from which the tizanidine is released and adjacent oral mucosa. Acidulants are approved or generally recognized as safe (GRAS) excipients for use in oral drug administration. Any approved or safe organic acid is suitable, such as ascorbic acid, benzoic acid, citric acid, fumaric acid, lactic acid, malic acid, sorbic acid and tartaric acid. A preferred acidulant is citric acid.

The amount of acidulant that is effective in any particular composition or dosage form will depend upon many factors such as the intended rate of release of the drug, the choice of acidulant, the rate at which it is released into the mouth and even the profundity of the patient's salivation. One method is to sample the pH of the patient's saliva coating the dosage form or pharmaceutical composition and see whether it is within the 2-7 pH range, or, yet more preferred, within the range of 2-5. Such routine experimentation is not considered to be undue.

One preferred embodiment of a pharmaceutical composition of tizanidine is a liquid that congeals when placed under the tongue or between cheek and gum. The congealed liquid is a mucoadhesive solid or semi-solid that slowly releases tizanidine over time. This embodiment possesses the advantage that the gelled composition conforms to the surfaces of the mouth, giving it a more comfortable feel. The liquid composition comprises a hydrophilic polymer selected from the group consisting of proteins, polysaccharides, cellulosic polymers and polyacrylates. Proteins include gelatin, hydrolyzed gelatin, albumin and collagen. Polysaccharides include pectin, carrageenan and alginic acid and their salts, guar gum, and tragacanth gum. Cellulosic polymers include hydroxyethylcellulose, hydroxypropylcellulose and hydroxypropylmethylcellulose. Preferred hydrophilic polymers are hydroxypropylcellulose and hydroxypropylmethylcellulose having a molecular weight range from 25000 to 2.5 million daltons. Such hydrophilic polymers are available under the trade names Klucel™ from the Hercules Corporation and Methocel™ from Dow Chemical Company. An alternative embodiment of this pharmaceutical composition comprises solutions of polymers having reverse thermal gellation (gel upon heating instead of upon cooling). Examples of such polymers are methylcellulose, triblock poly(lactide-co-glycolide) polyethylene glycol copolymers described in U.S. Pat. Nos. 6,004,573; 6,117,949 and 6,201,072, and thermosensitive biodegradable polymers based on poly(ether-ester)block copolymers as described in U.S. Pat. No. 5,702,717.

When the liquid congeals, the polymer chains cross-link by hydrogen bonding through tannic acid, which also is present in the composition, or alternative, comparably effective poly-protic cross-linking agent. The preferred cross-linking agent is USP tannic acid. Although liquid pharmaceutical compositions for buccal administration of drugs containing a polymer and tannic acid are described in International Publication No. WO 99/04764, of which we are inventors, that publication does not teach or suggest the use of such ingredients in a pharmaceutical composition adapted to deliver tizanidine sublingually or buccally.

Preferred liquid formulations that congeal in the mouth comprise about 0.1 wt. % to about 0.5 wt. % tizanidine, from about 0.1 wt % to about 5 wt. % of a hydrophilic polymer and from about 0.1 wt % to about 0.5 wt % of tannic acid with the remainder of the composition being made up of solvent, which is preferably water, ethanol and mixtures thereof, and other excipients such as colorants, flavorants, tonicity modifiers, viscosity modifiers, preservatives and the like. Note that congealing liquid compositions that contain tannic acid within the preferred amounts generally do not require a separate acidulant since the tannic acid functions both as a hydrogen bonding cross-linking agent and an acidulant.

An especially preferred dosage form of this invention is a tablet formed by multiple compression steps into an inner tablet core containing tizanidine surrounded by an annular body. The advantage of this tablet configuration is that the tizanidine-containing portion of the tablet is protected from disintegration by handling and, once in use, by mastication.

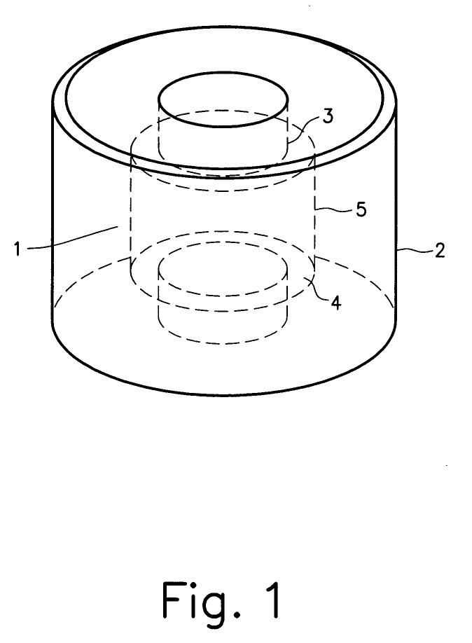

Referring to FIG. 1, the protected dosage form comprises a core tablet containing tizanidine sheathed in an annular body comprised of compressed powder or granular material. The core tablet has first and second opposed surfaces and a circumferential surface. “Sheathing” means that the annular body encircles the core tablet and is in contact with the core tablet about its circumferential surface, but leaves opposed surfaces of the core tablet substantially exposed. Core tablet 1 containing the tizanidine is recessed in the annular body 2. Core tablet 1 has opposed first and second surfaces 3 and 4 and an outer circumferential surface 5 extending between the opposed surfaces. Core tablet 1 is preferably cylindrical or disk shaped for ease of manufacture, but need not be so. The maximum distance across either of the opposed surfaces 3 or 4 is preferably from about 2 mm to about 12 mm, more preferably from about 4 mm to about 7 mm, most preferably about 5 mm. Opposed surfaces 3 and 4 can be flat, concave or convex and are preferably flat.

In outer contour, annular body 2 is preferably cylindrically shaped, but it can have any cross section, such as oval, elliptical or oblong. The outer diameter is preferably of from about 5 mm to about 15 mm, more preferably of from about 7 mm to about 12 mm, most preferably about 9 mm. The inner diameter can be any size up to about 2 mm less than the outer diameter. Preferably, the inner diameter is 3 mm or greater.

The solid dosage forms with a drug-containing core tablet sheathed in a compressed annular body of excipients can be produced using a novel set of tooling that is described in U.S. patent application Ser. No. 10/419,536, filed on Apr. 21, 2003 and PCT Application No. PCT/US02/36081, filed on Nov. 12, 2003 and published on Jul. 17, 2003 as International Patent Publication No. WO 03/057136, which are hereby incorporated by reference in their entirety, or by other multicompression techniques known in the art.

The core tablet can be formulated for any desired release profile, such as immediate release, delayed release, burst or pulsed release, sustained or zero order release, but most preferably immediate release. For immediate release, the core preferably contains a disintegrant like crospovidone to accelerate release. Other preferred excipients for an immediate release core tablet are α-lactose monohydrate, microcrystalline cellulose, sodium saccharine, and magnesium stearate. A preferred composition for the core tablet contains about 1-10 parts tizanidine hydrochloride, 50-70 parts α-lactose, 10-20 parts microcrystalline cellulose, about 0.1 to 1 part sodium saccharine and 15-25 parts crospovidone, exclusive of other excipients that may be present. The core tablet also may contain the acidulant.

The annular body can be formulated with any desired purpose in mind, such as taste masking. It also can contain the acidulant. The annular body can be formed of any pharmaceutically acceptable excipients. In particular, it may be mentioned that diluents, binders, disintegrants, glidants, lubricants, flavorants, colorants and the like can be included in the annular body. Blending and granulation with conventional excipients is well within the knowledge of those skilled in the art of tableting.

Preferred excipients for forming the annular body include hydroxypropyl cellulose (e.g., Klucel®), hydroxypropyl methylcellulose (e.g. Methocel®), microcrystalline cellulose (e.g., Avicel®), starch, lactose, sugars, compressible sugar, crospovidone (e.g. Kollidon™), polyvinylpyrrolidone (e.g. Plasdone®) and calcium phosphate. Yet more preferred excipients for forming the annular body are α-lactose monohydrate, microcrystalline cellulose and compressible sugar, An especially preferred ring excipient is a spray dried mixture of about 75% α-lactose monohydrate and 25% microcrystalline cellulose with a particle size distribution of d(15)<32 μm and d(90)<250 μm. Such a mixture is commercially available from Meggle AG, Wasserburg, Germany, under the tradename Microcellac™. Compressible sugar is available under the tradename Nu-Tab™ from CHR. Hansen, Hørsholm, Denmark.

A preferred composition of the annular body is about 45-50 parts compressible sugar, about 30-40 parts α-lactose monohydrate, 1-10 parts microcrystalline cellulose, and 1-10 parts crospovidone.

Having described the invention with reference to particular preferred embodiments, the invention will now be further illustrated by the following example which is for illustrative purposes only and not intended to limit the invention.

### EXAMPLE

Sublingual Tablet Preparation

The sublingual tablets used in this study were formed into an inner core of a fast disintegrating formulation containing 2 mg tizanidine and an outer annular body of protective excipients.

The inner cores were made by mixing 4.5 parts tizanidine hydrochloride and 20 parts crospovidone for 2 minutes. One half part sodium saccharin, 73.6 parts of Microcellac 100™, and 0.4 parts menthol were added and the mixing was continued for 3 more minutes. One part magnesium stearate was added and the mixing was continued for a half a minute. This mixture was compressed on a Manesty f3 tablet press fitted with a five mm flat beveled punch. The tablets formed were of 5 mm diameter, weighed 45 mg each, were about 2 mm thick and had a hardness of 1-3.5 Kp.

The outer annular body was made by mixing 48.5 parts Nu-Tab™, 45 parts of Microcellac 100™, 0.5 parts of sodium saccharin and 5 parts of crospovidone for 5 minutes, adding one part magnesium stearate, mixing for another half of a minute, and then compressing on a Manesty f3 tablet press fitted with a set of tooling like that described in U.S. patent application Ser. No. 10/419,536, filed on Apr. 21, 2003 and International Patent Publication No. WO 03/057136. The entire tablet weight was 290 mg. The outer diameter was 9 mm. The tablet height about 4.5 mm and the hardness was 5-9 Kp.

Pharmacokinetic Trial

Twelve volunteer subjects were administered a 4 mg commercial oral preparation of tizanidine (Zanaflex™) and the 2 mg sublingual tablet described here in a crossover study. Two groups were randomized and there was a one week washout period between administrations. The volunteers were in the fasted state when the drugs were administered. The sublingual tablets were placed under the tongue for 5 minutes and tablet remnants, if any, were swallowed. The oral formulation was administered with a glass of water. Blood samples were taken at 0, 0.5, 1.0, 1.5, 2.0, 2.5, 3.0, 4.0, 5.0, 6.0 and 7.0 hours after administration. The plasma was separated from the whole blood and the tizanidine concentration was determined by a validated HPLC assay. The samples were blinded from the analysts. All twelve volunteers participated in the sublingual arm while one volunteer did not participate in the oral delivery arm.

Results

Table 1 collects the results of analyses of tizanidine in plasma for twelve test subjects who were administered 2 mg tizanidine in a sublingual formulation.

TABLE 1
Plasma Tizanidine Levels (ng/g) after Sublingual Delivery of 2 mg Tizanidine

| Time | | | | | | | | | | | | |
|---|---|---|---|---|---|---|---|---|---|---|---|---|
| After | | | | | | | | | | | | |
| Dosing | Test Subject No. | | | | | | | | | | | |
| (h) | 1 | 2 | 3 | 4 | 5 | 6 | 7 | 8 | 9 | 10 | 11 | 12 |
| 0 | <98.40 | NRV^{a} | <98.40 | <98.40 | <98.40 | <98.40 | <98.40 | <98.40 | <98.40 | <98.40 | <98.40 | <98.40 |
| 0.5 | 775.02 | 532.00 | 1553.79 | 145.17 | 1896.26 | 776.45 | 443.45 | 1431.47 | 313.12 | 1961.88 | 471.87 | 245.53 |
| 1 | 1278.80 | 988.08 | 1459.29 | 481.79 | 2019.72 | 1197.35 | 1034.11 | 1973.34 | 567.10 | 1431.73 | 973.92 | 952.86 |
| 1.5 | 972.00 | 924.2 | 998.88 | 513.86 | 1691.20 | 824.94 | 1845.35 | 1963.51 | 741.25 | 1079.73 | 1055.64 | 1021.82 |
| 2 | 788.93 | NRV | 990.63 | 766.84 | 1618.61 | 548.21 | 1832.81 | 1471.67 | 1880.60 | 995.41 | 660.18 | 675.70 |
| 2.5 | 560.71 | 643.96 | 838.46 | 639.48 | 935.25 | 390.09 | 1721.56 | 968.31 | 1025.96 | 609.41 | 414.75 | 506.19 |
| 3 | 341.03 | 467.46 | 758.47 | 471.44 | 874.76 | 275.99 | 1447.37 | 650.90 | 585.00 | 519.01 | 301.19 | 294.73 |
| 4 | 245.64 | 375.05 | 472.62 | 308.53 | 497.04 | 170.75 | 866.4 | 403.68 | 357.36 | 264.30 | 152.11 | 162.55 |
| 5 | 110.77 | 244.92 | 282.94 | 323.35 | 304.18 | 137.31 | 749.04 | 243.47 | 270.23 | 171.07 | <98.40 | 116.40 |
| 6 | <98.40 | NRV | NRV | 145.36 | 253.06 | <98.40 | 489.89 | 184.65 | 183.24 | 118.64 | <98.40 | <98.40 |
| 7 | <98.40 | 101.95 | 117.57 | <98.40 | 183.79 | <98.40 | 389.01 | 100.18 | <98.40 | <98.40 | <98.40 | <98.40 |

^{a}NRV = No value reported

Table 2 collects the data for 11 of the same twelve test subjects (test subject 6 did not participate in this arm of the trial) who were administered 4 mg tizanidine in a standard commercial oral formulation.

TABLE 2
Plasma Tizanidine Levels (ng/g) After Gastric Delivery of 4 mg Tizanidine in a
Commercial Immediate Release Formulation

| Time | | | | | | | | | | | |
|---|---|---|---|---|---|---|---|---|---|---|---|
| After | | | | | | | | | | | |
| Dosing | Test Subject No. | | | | | | | | | | |
| (h) | 1 | 2 | 3 | 4 | 5 | 7 | 8 | 9 | 10 | 11 | 12 |
| 0 | <98.40 | <98.40 | <98.40 | <98.40 | <98.40 | <98.40 | <98.40 | <98.40 | <98.40 | <98.40 | <98.40 |
| 0.5 | 111.53 | NRV^{a} | 2750.73 | 143.68 | 375.93 | 1581.27 | 1442.94 | 417.87 | 1680.10 | 2336.94 | 812.94 |
| 1 | 1263.88 | 1149.86 | 2164.91 | 525.09 | 5715.05 | 2248.04 | 3417.33 | 2513.27 | 1515.56 | 1395.25 | 1504.56 |
| 1.5 | 1263.59 | 1673.44 | 1608.80 | 873.90 | 3990.80 | 2991.92 | 2971.57 | 1306.68 | 1181.31 | 1058.19 | 1344.10 |
| 2 | 817.28 | 1934.76 | 1282.80 | 1086.72 | 3111.55 | 2471.10 | 2829.05 | 890.87 | 782.03 | 847.13 | 1150.98 |
| 2.5 | 711.36 | 1406.92 | 970.04 | 1138.61 | 2224.19 | 3448.21 | 3164.19 | 672.94 | 517.53 | 490.23 | 823.30 |
| 3 | 434.83 | 965.01 | 818.19 | 447.45 | 1787.40 | 2802.43 | 2016.60 | 419.12 | 360.95 | 454.67 | 523.06 |
| 4 | 198.21 | 577.89 | 477.16 | 305.11 | 1348.44 | 1933.08 | 1121.49 | 197.08 | 218.69 | 316.96 | 336.86 |
| 5 | 170.07 | NRV | 301.57 | 272.98 | 962.62 | 1209.45 | 964.26 | 180.33 | 161.26 | 230.63 | 159.62 |
| 6 | <98.40 | NRV | 292.12 | <98.40 | 438.65 | 704.52 | 580.47 | <98.40 | <98.40 | 147.53 | 99.22 |
| 7 | <98.40 | <98.40 | 150.48 | 108.18 | 464.21 | 363.02 | 212.05 | <98.40 | <98.40 | <98.40 | <98.40 |

^{a}NRV = No value reported

Table 3 collects the calculated pharmacokinetic parameters for both groups.

TABLE 3
Summary of Pharmacokinetic Data for 2 mg Sublingual
(test) vs. 4 mg Oral (ref)

| | AUC | AUC_{inf} | t_{1/2} | Tₘₐₓ | Cₘₐₓ |
|---|---|---|---|---|---|
| Test Subject No. | (h * ng/g) | (h * ng/g) | (h) | (h) | (ng/g) |
| 1-test | 2799.9 | 2799.9 | 1.1 | 1.0 | 1278.8 |
| 2-test | 3110.7 | 3393.0 | 1.9 | 1.0 | 988.1 |
| 3-test | 4503.7 | 4783.8 | 1.7 | 0.5 | 1553.8 |
| 4-test | 2404.4 | 2404.4 | 1.9 | 2.0 | 766.8 |
| 5-test | 5882.8 | 6366.6 | 1.8 | 1.0 | 2019.7 |
| 6-test | 2383.6 | 2383.6 | 1.5 | 1.0 | 1197.4 |
| 7-test | 6824.0 | 8040.4 | 2.2 | 1.5 | 1845.4 |
| 8-test | 5274.2 | 5483.9 | 1.5 | 1.0 | 1973.3 |
| 9-test | 3513.6 | 3513.6 | 1.8 | 2.0 | 1880.6 |
| 10-test | 3982.3 | 3982.3 | 1.3 | 0.5 | 1961.9 |
| 11-test | 2166.2 | 2166.2 | 1.0 | 1.5 | 1055.6 |
| 12-test | 2201.0 | 2201.0 | 1.1 | 1.5 | 1021.8 |
| 1-ref | 2778.2 | 2778.2 | 1.2 | 1.0 | 1263.9 |
| 2-ref | | | | 2.0 | 1934.8 |
| 3-ref | 6148.4 | 6536.5 | 1.8 | 0.5 | 2750.7 |
| 4-ref | 2851.8 | 3289.6 | 2.8 | 2.5 | 1138.6 |
| 5-ref | 12031.1 | 13246.5 | 1.8 | 1.0 | 5715.1 |
| 7-ref | 12500.7 | 13153.8 | 1.2 | 2.5 | 3448.2 |
| 8-ref | 11197.2 | 11607.9 | 1.3 | 1.0 | 3417.3 |
| 9-ref | 3592.6 | 3592.6 | 1.1 | 1.0 | 2513.3 |
| 10-ref | 3488.9 | 3488.9 | 1.3 | 0.5 | 1680.1 |
| 11-ref | 4100.0 | 4100.0 | 1.8 | 0.5 | 2336.9 |
| 12-ref | 3805.9 | 3805.9 | 1.2 | 1.0 | 1504.6 |
| AVG (test) | 3753.9 | 3959.9 | 1.6 | 1.3 | 1461.9 |
| AVG (ref) | 6249.5 | 6560.0 | 1.6 | 1.2 | 2518.5 |
| geomn (test) | 3481.9 | 3608.7 | 1.5 | 1.1 | 1391.6 |
| geomn (ref) | 5254.5 | 5462.0 | 1.5 | 1.0 | 2254.8 |
| s.e. (test) | 451.4 | 540.2 | 0.1 | 0.1 | 132.7 |
| s.e. (ref) | 1162.1 | 1256.6 | 0.1 | 0.2 | 382.2 |
| s.d. (test) | 1564 | 1871 | 0.4 | 0.5 | 460 |
| s.d. (ref) | 4026 | 4353 | 0.5 | 0.8 | 1324 |
| r.s.d. (test) | 0.4166 | 0.4725 | — | — | — |
| r.s.d. (ref) | 0.6442 | 0.6636 | — | — | — |
| Δ % r.s.d | −35.3 | −28.8 | — | — | — |

The average total amount absorbed (the area under the plasma concentration vs. time curve extrapolated to infinity (AUC_{inf)) was} 6560 for the 4 mg oral tablet while the result was 3960 for the 2 mg sublingual tablet. Normalizing for dose gives 1640/mg for the oral delivery and 1980/mg for the sublingual delivery, reflecting a 20% increase in bioavailability. The average Cₘₐₓ for the 2 mg sublingual delivery was 1462 (731/mg) while for the 4 mg oral dose it was 2519 (630/mg) or about 16% higher. The standard deviation of the AUC for the oral formulation was 4353 (relative standard deviation of 66%) while the standard deviation of the data for the 2 mg sublingual formulation was 1871 (relative standard deviation of 47%) reflecting a decrease in variation of 28.8%. Therefore, we have shown by this study that sublingual and buccal delivery gives less variable results and improved bioavailability compared to conventional oral delivery in which the drug is absorbed in the intestine.

Although this invention has been described with respect to certain specific embodiments, it will be appreciated by those skilled in the art that various modifications may be made without departing from the spirit and scope of the invention as defined by the claims that follow.

What is claimed is:

1. A method of treating muscle spasms comprising administering an effective anti-spasmodic amount of tizanidine by a route of administration selected from the group consisting of buccal administration and sublingual administration.

2. The method of claim 1 wherein the tizanidine is administered in a pharmaceutical composition or dosage form that releases 80% or more of the tizanidine in 20 minutes or less.

3. The method of claim 2 wherein the pharmaceutical composition or dosage form releases 80% or more of the tizanidine in 5 minutes or less.

4. A method of increasing the bioavailability of tizanidine by administration of an effective anti-spasmodic amount of tizanidine by a route selected from the group consisting of buccal administration and sublingual administration.

5. The method of claim 4 wherein the increase in bioavailability is an increase of 10% or more of the average area under the curve extrapolated to infinity of the plasma concentration of tizanidine over time of a first population patients who are administered tizanidine sublingually or buccally compared to a second population of patients who are administered an equivalent dose of tizanidine by swallowing an immediate release tablet.

6. The method of claim 5 wherein the first population and the second population are the same and the time that tizanidine is administered by swallowing an immediate release tablet is separated from the time that tizanidine is administered sublingually or buccally by a washout period.

7. The method of claim 5 wherein the increase in bioavailability is an increase of 20% or more.

8. The method of claim 5 wherein the immediate release tablet comprises the excipients colloidal silicon dioxide, stearic acid, microcrystalline cellulose and anhydrous lactose.

9. The method of claim 8 wherein the immediate release tablet is ZANAFLEX™.

10. A method of reducing variations in the bioavailability of tizanidine between individuals in a patient population receiving tizanidine therapy by administration of tizanidine by a route selected from the group consisting of buccal administration and sublingual administration.

11. The method of claim 10 wherein the patient population is the patients receiving tizanidine therapy at a single health care facility.

12. The method of claim 11 wherein the population includes a proportion of the population who have been administered tizanidine orally but do not respond well and are subsequently administered tizanidine sublingually or buccally and wherein the reduction in variation among the population is improvement in the suppression of muscle spasms of the proportion of the population that does not respond well to orally administered tizanidine as evidenced by observations of health care personnel or medical records.

13. The method of claim 11 wherein the patient population is the patients receiving tizanidine therapy from a single doctor.

14. The method of claim 13 wherein the population includes a proportion of the population who have been administered tizanidine orally but do not respond well and are subsequently administered tizanidine sublingually or buccally and wherein the reduction in variation among the population is improvement in the suppression of muscle spasms of the proportion of the population that does not respond well to orally administered tizanidine as evidenced by observations of health care personnel or medical records.

15. The method of claim 10 wherein the bioavailability is measured by the area under the curve of blood plasma over time extrapolated to infinity (AUC_{inf}) and the reduction in variation in bioavailability is measured using the relative standard deviation of AUC_{inf}.

16. The method of claim 15 wherein the reduction is about 10% or more.

17. The method of claim 16 wherein the reduction is about 20% or more.

18. The method of claim 17 wherein the reduction is about 30% or more.

19. A tizanidine pharmaceutical composition or oral dosage form especially adapted to release tizanidine in the mouth comprising tizanidine and a pharmaceutically acceptable carrier.

20. The tizanidine pharmaceutical composition or oral dosage form of claim 19 further comprising an acidulant.

21. The tizanidine pharmaceutical composition or oral dosage form of claim 20 wherein the acidulant is selected from the group consisting of ascorbic acid, benzoic acid, citric acid, fumaric acid, lactic acid, malic acid, sorbic acid and tartaric acid.

22. The tizanidine pharmaceutical composition or oral dosage form of claim 21 wherein the acidulant is citric acid.

23. The tizanidine pharmaceutical composition or oral dosage form of claim 19 wherein 80% of the tizanidine is released in twenty minutes or less after being taken into the mouth.

24. The tizanidine pharmaceutical composition or oral dosage form of claim 23 wherein 80% of the tizanidine is released in five minutes or less after being taken into the mouth.

25. The tizanidine pharmaceutical composition or oral dosage form of claim 19 that is a congealing liquid pharmaceutical composition comprising a hydrophilic polymer and a poly-protic hydrogen bonding cross-linking agent.

26. The tizanidine pharmaceutical composition of claim 25 wherein the cross-linking agent is tannic acid.

27. The tizanidine pharmaceutical composition of claim 25 wherein the hydrophilic polymer is selected from the group consisting of proteins, polysaccharides, cellulosic polymers and polyacrylates.

28. The tizanidine pharmaceutical composition of claim 27 wherein the protein is selected from the group consisting of gelatin, hydrolyzed gelatin, albumin and collagen.

29. The tizanidine pharmaceutical composition of claim 27 wherein the cellulosic polymer is selected from the group consisting of hydroxyethylcellulose, hydroxypropylcellulose and hydroxypropylmethylcellulose.

30. The tizanidine pharmaceutical composition of claim 27 wherein the polysaccharides is selected from the group consisting of pectin, carrageenan, alginic acid and their salts, guar gum and tragacanth gum.

31. The tizanidine pharmaceutical composition or oral dosage form of claim 19 that comprises a core tablet containing tizanidine sheathed in an annular body of pharmaceutical excipients.
